(19) United States
(12) Patent Application Publication
(10) Pub. No.: US 2009/0305940 A1
(43) Pub. Date: Dec. 10, 2009

(54) Washing amd cleaning agents containing alcohol ethoxylate propoxylates

(76) Inventors: Guenther Schimmel, Erftstadt (DE); Hans Juergen Scholz, Alzenau (DE); Johannes Himmrich, Eppstein (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/919,026

(22) PCT Filed: Apr. 15, 2006
(86) PCT No.: PCT/EP2006/003489

§ 371 Date: Aug. 6, 2009

(30) Foreign Application Priority Data
Apr. 21, 2005 (DE) 10 2005 018 501.0

(51) Int. Cl. C11D 1/722; C07C 43/13; C11D 1/825

(52) U.S. Cl. 510/421; 568/625

(57) ABSTRACT

The invention relates to washing and cleaning agents that, as a washing active substance, contain alcohol ethoxylate propoxylates, which are obtained by the addition of ethylene oxide and propylene oxide to fatty alcohols, the number of the added propylene oxide units needing to be greater than the number of the added ethylene oxide units.

The invention relates to the use of alcohol polyethoxylate polypropoxylates for producing low-foam washing and cleaning agents with improved cleaning power toward oily-greasy soilings and stains.

The use of alcohol alkoxylates as nonionic surfactant in washing and cleaning agents is known. The publication “Comunicaciones presentadas a la Jornadas del Comite Espanol de la Detergencia (1989)”, 20, 45-61, CODEN: CJCDD7, ISSN: 0212-7466 describes alcohol ethoxylates as low-foam surfactants, and the good soil release capability of alcohol polyethoxylate polypropoxylates with a high EO content. EP 694 606 claims cleaning agents for hard surfaces containing fatty alcohol ethoxylate propoxylates having 1 to 10 EO and 1 to 10 PO units as foam-suppressing agent.

High demands are placed on modern washing and cleaning agents. Besides a good and rapid cleaning power toward dirt and grease, they must be use-friendly, exhibit high detergency even at low washing temperatures, be gentle to colors and textile fibers, and also be environmentally compatible.

The object was to provide mild, low-foam surfactants which exhibit improved washing and cleaning power, are easy to formulate and process and do not have a tendency to form gels even at low washing temperatures and if the amount of water introduced is small.

Surprisingly, it has been found that this object is achieved by alcohol ethoxylate propoxylates wherein the propoxylate content is greater than the ethoxylate content.

The invention provides washing and cleaning agents containing alcohol ethoxylate propoxylates of formula 1

R¹-A-OR² (1)

in which R¹ is C₆-C₃₀-alkyl or C₆-C₃₀-alkenyl,

A is a group of the formula

—(OC₂H₄)ₓ—(OC₃H₆)_{y}— or —(OC₃H₆)_{y}—(OC₂H₄)ₓ—,

R² is hydrogen or C₁-C₈-alkyl, x is a number from 1 to 10 and y is a number from 2 to 12, with the proviso that the value of the index y is always higher than the value of the index x.

R¹ is a linear or branched alkyl or alkenyl group and contains preferably 12 to 22, in particular 12 to 18, carbon atoms. R² is preferably hydrogen, but can also be methyl, ethyl, n-propyl, n-butyl, n-pentyl, n-hexyl, n-heptyl or n-octyl.

The compounds of formula 1 can have a narrow homolog distribution of the ethylene oxide or propylene oxide (“narrow range ethoxylates-propoxylates”) or a broad homolog distribution of the ethylene oxide or propylene oxide (“broad range ethoxylates-propoxylates”).

For the content of ethylene oxide (index x), the following ranges are preferred: 1 to 8, 2 to 6, 2 to 4. For the content of propylene oxide (index y), the following ranges are preferred: 2 to 10, 3 to 7 and 4 to 6. The sum of numbers for x and y is preferably 3 to 14, 4 to 13 and 5 to 12. It is essential that in a specific case, the value for the index y must be greater than the value for the index x. The EO and PO units can be arranged in random distribution or in blocks, the latter variant being preferred.

The stated degrees of ethoxylation and degrees of propoxylation are statistical average values, which may be an integer or a fraction for a specific product.

In one preferred embodiment of the invention, the washing and cleaning agents according to the invention contain one or more alcohol ethoxylate propoxylates according to formula 1, where R¹ is a linear or branched alkyl group having 12 to 18 carbon atoms, preferably a linear or branched radical from alcohols of native origin having 12 to 18 carbon atoms, and technical-grade mixtures thereof, which are produced, for example, during the high-pressure hydrogenations of technical-grade methyl esters based on fats and oils or aldehydes from the Roelen oxo synthesis, and as monomer fraction in the dimerization of unsaturated fatty alcohols, for example from lauryl, coconut, palm fat, palm kernel, stearyl, isostearyl, oleyl, caproic, caprylic, capric, 2-ethylhexyl, isotridecyl, myristyl, cetyl, elaidyl, petroselinyl, arachyl, gadoleyl, behenyl, erucyl, brassidyl alcohol or from synthetic alcohols, for example Guerbet alcohol.

A is a group of the formula —(OC₃H₆)_{y}—(OC₂H₄)ₓ—, the index x is a number from 1 to 10, preferably 1 to 8, particularly preferably from 2 to 6 and extraordinarily preferably from 2 to 4, the index y is a number from 1 to 12, preferably 2 to 10 particularly preferably from 3 to 7 and extraordinarily preferably from 4 to 6,

and the sum of the numbers x+y is in the range from 2 to 20, preferably 3 to 14, particularly preferably 4 to 13 and extraordinarily preferably 5 to 12, and R² is hydrogen.

In a particularly preferred embodiment of the invention, the washing and cleaning agents according to the invention contain one or more alcohol ethoxylate propoxylates according to formula 1, where R¹ is a linear or branched alkyl group having 12 to 18 carbon atoms, or mixtures thereof, preferably a C_{12/15}-alkyl group, A is a group of the formula —(OC₃H₆)_{y}—(OC₂H₄)ₓ—, the index x is a number from 1 to 4, preferably 2, the index y is a number from 4 to 10, preferably 4 to 8, particularly preferably from 5 to 7 and extraordinarily preferably 5,

and the sum of the numbers x+y is in the range from 5 to 14, preferably 6 to 10, particularly preferably 7 to 9 and extraordinarily preferably 7 to 8, and R² is hydrogen.

In an extraordinarily preferred embodiment of the invention, the washing and cleaning agents according to the invention contain a C_{12/15}-alcohol propoxylate ethoxylate with 5 propoxy groups and 2 ethoxy groups, where the EO and PO units are arranged in blocks, prepared by propoxylation of oxo alcohol with 5 mol equivalents of PO and subsequent ethoxylation of the C_{12/15}-fatty alcohol propoxylate (5 PO) with 2 mol equivalents of EO. This fatty alcohol propoxylate ethoxylate used particularly preferably according to the invention is available under the trade name ®Genapol EP 2552 from Clariant GmbH.

In a further exceptionally preferred embodiment of the invention, the washing and cleaning agents according to the invention contain C₆-C₁₄-alcohol propoxylate ethoxylates having 2 to 6 mol of EO and 4 to 8 mol of PO. These products are available under the trade names ®Genapol EP 6068, 2424, 2445 from Clariant GmbH.

According to the invention, preference is given to using compounds according to formula 1 whose melting point is below +10° C., preferably below +3° C., particularly preferably below 0° C., especially preferably below −10° C.

The alcohol ethoxylate propoxylates used according to the invention in washing agents can be prepared in accordance with known methods by ethoxylation and propoxylation of the corresponding alcohols of formula R¹—OH. For this, the fatty alcohol or oxo alcohol is initially introduced, alkalized with 50% strength NaOH and then, at about 130° C., successively propoxylated, then ethoxylated or ethoxylated then propoxylated, and, when the reaction is complete, adjusted to a pH of from 6 to 8, for example with isononanoic acid. In this way, compounds of formula 1 where R²═H are obtained. If R² is to be alkyl, the compounds of formula 1 where R¹═OH are etherified in a manner known per se with an alcohol of the formula R²—OH.

The washing and cleaning agents according to the invention can be present in solid form as powder, granules, tablet, or in liquid form as aqueous, aqueous/organic, aqueous/alcoholic or organic formulations. Further embodiments may be: emulsions, dispersions, gels and suspensions.

The washing and cleaning agents according to the invention containing the abovementioned alcohol ethoxylate propoxylates may be of varying nature. Preference is given to solid washing agents, in particular standard washing agents for white and colored laundry and light-duty washing agents for the machine and for handwashing. Preference is likewise given to liquid washing agents, dishwashing agents for the machine, hand dishwashing agents, liquid all-purpose cleaners, bath cleaners, glass cleaners, and floor cleaners.

A preferred embodiment of the invention is solid washing agents, as powder washing agents, granule washing agents or in the form of tablets, containing the abovementioned alcohol ethoxylate propoxylates in amounts by weight of from 0.5 to 10%, preferably 1 to 8%, particularly preferably 3 to 7%.

A further preferred embodiment of the invention is liquid washing agents containing the abovementioned fatty alcohol ethoxylate propoxylates in amounts by weight of from 0.5 to 10%, preferably 1 to 8%, particularly preferably 3 to 6%.

A further preferred embodiment of the invention is hand dishwashing agents containing the abovementioned fatty alcohol ethoxylate propoxylates in amounts by weight of from 0.5 to 10%, preferably 1 to 7%, particularly preferably 1 to 5%.

A further preferred embodiment of the invention is all-purpose cleaners containing the abovementioned fatty alcohol ethoxylate propoxylates in amounts by weight of from 0.5 to 10%, preferably 1 to 7%, particularly preferably 1 to 5%.

In a preferred embodiment, the washing, cleaning agents according to the invention contain a further nonionic surfactant from the class of alcohol ethoxylates and/or noninventive alcohol-EO/PO adducts with a high EO content and a low PO content.

Suitable alcohol ethoxylates are fatty alcohol or oxo alcohol ethoxylates of formula 2

R¹—O—(CH₂CH₂O)ₙ—R² (2)

where

R¹ is a linear or branched, saturated or unsaturated alkyl group having 6 to 22 carbon atoms, preferably 8 to 18 carbon atoms, and n is numbers from 1 to 50, preferably 4 to 25, particularly preferably 4 to 20.

Examples are the adducts of, on average, 1 to 50, preferably 4 to 25, particularly preferably 4 to 20, mol of ethylene oxide onto caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol, and technical-grade mixtures thereof which are produced, for example, during the high-pressure hydrogenation of technical-grade methyl esters based on fats and oils or aldehydes from the Roelen oxo synthesis, and as monomer fraction in the dimerization of unsaturated fatty alcohols. Preference is given to adducts of from 4 to 25 mol of ethylene oxide onto technical-grade fatty alcohols having 12 to 18 carbon atoms, such as, for example, coconut, palm, palm kernel or tallow fatty alcohol. This surfactant class also includes the Genapol™ grades from Clariant GmbH.

The washing and cleaning agents according to the invention can additionally also contain alcohol-EO/PO adducts with a high EO content and a low PO content.

Examples are the adducts of, on average, 1 to 50, preferably 4 to 25, particularly preferably 4 to 20, mol of ethylene oxide and 1 to 20, preferably 2 to 10, particularly preferably 2 to 6, mol of propylene oxide onto the abovementioned fatty alcohol or oxo alcohols.

The washing and cleaning agents according to the invention can contain further surfactants of a nonionic, anionic, cationic or amphoteric nature, and customary auxiliaries and additives in varying amounts.

Suitable further nonionic surfactants are polyethylene, polypropylene and polybutylene oxide adducts of alkylphenols having 6 to 12 carbon atoms in the alkyl chain, addition products of ethylene oxide with a hydrophobic base, formed from the condensation of propylene oxide with propylene glycol or addition products of ethylene oxide with a reaction product of propylene oxide and ethylenediamine.

Furthermore, semipolar nonionic surfactants can be used, for example amine oxides of formula IV

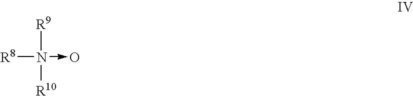

in which R⁸ is an alkyl, hydroxyalkyl or alkylphenol group or mixtures thereof with a chain length of from 8 to 22 carbon atoms; R⁹ is an alkylene or hydroxyalkylene group having 2 to 3 carbon atoms or mixtures thereof; R¹⁰ is an alkyl or hydroxyalkyl group having 1 to 3 carbon atoms or a polyethylene oxide group having 1 to 3 ethylene oxide units. The R¹⁰/R⁹ groups can be joined together via an oxygen or nitrogen atom and thus form a ring.

These amine oxides comprise particularly C₁₀-C₁₈-alkyldimethylamine oxides and C₈-C₁₀-alkoxyethyldihydroxyethylamine oxides.

Instead of or in addition to the nonionic surfactants, the mixtures according to the invention can also contain anionic surfactants.

Suitable anionic surfactants are primarily straight-chain and branched alkyl sulfates, alkylsulfonates, alkyl carboxylates, alkyl phosphates, alkyl ester sulfonates, arylalkylsulfonates, alkyl ether sulfates and mixtures of the specified compounds. In the text below, some of the types of anionic surfactants under consideration will be described in more detail.

Preference is given to secondary alkanesulfonates. Secondary alkane-sulfonates are surfactants of the formula R—SO₃M whose alkyl group R is saturated or unsaturated, linear or branched and which can also carry hydroxyl groups, where the terminal carbon atoms of the alkyl chain have no sulfonate group. Preference is given to secondary alkanesulfonates with linear alkyl groups having 9 to 25 carbon atoms, preferably having 10 to 20 carbon atoms, particularly preferably having 13 to 17 carbon atoms. Counterion M can be sodium, potassium, ammonium, mono-, di- or trialkanolammonium, calcium, magnesium ion or mixtures thereof. Preference is given to sodium salts of the secondary alkanesulfonates.

Also of suitability are alkyl ester sulfonates. Alkyl ester sulfonates are linear esters of C₈-C₂₀-carboxylic acids (i.e. fatty acids) which are sulfonated by SO₃, as described in “The Journal of the American Oil Chemists Society”, 52 (1975), pp. 323-329. Suitable starting materials are natural fatty derivatives such as, for example, tallow or palm oil fatty acid.

Use is likewise made of alkyl sulfates and alkyl ether sulfates. Alkyl sulfates are water-soluble salts or acids of the formula ROSO₃M, in which R is preferably a C₁₀-C₂₄-hydrocarbon radical, preferably an alkyl or hydroxyalkyl radical having 10 to 20 carbon atoms, particularly preferably a C₁₂-C₁₈-alkyl or hydroxyalkyl radical. M is hydrogen or a cation, e.g. an alkali metal cation (e.g. sodium, potassium, lithium) or ammonium or substituted ammonium, e.g. a methyl-, dimethyl- and trimethylammonium cation or a quaternary ammonium cation, such as tetramethylammonium and dimethylpiperidinium cation and quaternary ammonium cations, derived from alkylamines such as ethylamine, diethylamine, triethylamine and mixtures thereof. Alkane chains having C₁₂-C₁₆ are preferred here for low wash temperatures (e.g. below about 50° C.) and alkyl chains having C₁₆-C₁₈ are preferred for higher wash temperatures (above about 50° C.).

The alkyl ether sulfates are water-soluble salts or acids of the formula RO(A)ₘSO₃M, in which R is an unsubstituted C₁₀-C₂₄-alkyl or hydroxyalkyl radical having 10 to 24 carbon atoms, preferably a C₁₂-C₂₀-alkyl or hydroxyalkyl radical, particularly preferably a C₁₂-C₁₈-alkyl or hydroxyalkyl radical. A is an ethoxy or propoxy unit, M is a number greater than 0, typically between about 0.5 and about 6, particularly preferably between about 0.5 and about 3, and M is a hydrogen atom or a cation, such as, for example, a metal cation (e.g. sodium, potassium, lithium, calcium, magnesium, etc.), ammonium or a substituted ammonium cation. Examples of substituted ammonium cations are methyl-, dimethyl-, trimethylammonium and quaternary ammonium cations, such as tetramethylammonium and dimethylpiperidinium cations, and also those which are derived from alkylamines, such as ethylamine, diethylamine, triethylamine, mixtures thereof and the like. Examples which may be given are C₁₂-C₁₈-alkyl polyethoxylate(1.0) sulfate, C₁₂-C₁₈-alkyl polyethoxylate(2.25) sulfate, C₁₂-C₁₈-alkyl polyethoxylate(3.0) sulfate, C₁₂-C₁₈-alkyl polyethoxylate(4.0) sulfate, where the cation is sodium or potassium.

Other anionic surfactants which are useful for use in washing and cleaning agents are C₈-C₂₄-olefinsulfonates, sulfonated polycarboxylic acids, prepared by sulfonation of the pyrolysis products of alkaline earth metal citrates, as described, for example, in the British patent GB 1,082,179, alkylglycerol sulfates, fatty acyl glycerol sulfates, oleyl glycerol sulfates, alkylphenol ether sulfates, primary paraffinsulfonates, alkyl phosphates, alkyl ether phosphates, isethionates, such as acyl isethionates, N-acyltaurides, alkyl succinamates, sulfosuccinates, monoesters of sulfosuccinates (particularly saturated and unsaturated Cl₁₂-C₁₈-monoesters) and diesters of sulfosuccinates (particularly saturated and unsaturated C₁₂-C₁₈-diesters), acyl sarcosinates, sulfates of alkylpolysaccharides, such as sulfates of alkyl olyglycosides, branched primary alkyl sulfates and alkyl polyethoxycarboxylates, such as those of the formula RO(CH₂CH₂)ₖCH₂COO⁻M⁺, in which R is a C₈-C₂₂-alkyl, k is a number from 0 to 10 and M is a soluble salt-forming cation. Resin acids or hydrogenated resin acids, such as rosin or hydrogenated rosin or tall oil resins and tall oil resin acids can likewise be used. Further examples are described in “Surface Active Agents and Detergents” (Vol. I and II, Schwartz, Perry and Berch). A large number of such surfactants is also described in U.S. Pat. No. 3,929,678.

Examples of amphoteric surfactants which can be used in the formulations of the present invention are primarily those which are broadly described as derivatives of aliphatic secondary and tertiary amines in which the aliphatic radical may be linear or branched and in which one of the aliphatic substituents contains between 8 and 18 carbon atoms and contains an anionic, water-soluble group, such as, for example, carboxy, sulfonate, sulfate, phosphate or phosphonate.

Further preferred amphoteric surfactant are alkyldimethylbetaines, alkylamidobetaines and alkyldipolyethoxybetaines with an alkyl radical which be linear or branched, having 8 to 22 carbon atoms, preferably having 8 to 18 carbon atoms and particularly preferably having 12 to 18 carbon atoms. These compounds are marketed, for example, by Clariant GmbH under the trade name Genagen® CAB.

### Auxiliaries and Additives

Depending on the intended use, besides the specified surfactants, the washing and cleaning agents also contain the auxiliaries and additives specific in each case, for example builders, salts, bleaches, bleach activators, bleach catalysts, optical brighteners, complexing agents, graying inhibitors, solubility promoters, acidic additives, enzymes, thickeners, preservatives, fragrances and dyes, pearlizing agents, foam inhibitors, sequestrants.

Suitable organic and inorganic builders are neutral or in particular alkaline salts which are able to precipitate out calcium ions or bind them in complexes. Suitable and in particular ecologically acceptable builder substances, such as finely crystalline, synthetic water-containing zeolites of type NaA, which have a calcium binding capacity in the range from 100 to 200 mg CaO/g, are preferably used. In nonaqueous systems, preference is given to using sheet silicates. Zeolite and the sheet silicates can be present in the agent in an amount up to 20% by weight. Organic builder substances which can be used are, for example, the percarboxylic acids preferably used in the form of their sodium salts, such as citric acid and nitriloacetate (NTA), ethylenediaminetetraacetic acid, provided such a use is not objectionable on ecological grounds. Analogously to this, it is also possible to use polymeric carboxylates and salts thereof. These include, for example, the salts of homopolymeric or copolymeric polyacrylates, polymethyacrylates and in particular copolymers of acrylic acid with maleic acid, preferably those of 50% to 10% maleic and also polyvinylpyrrolidone and urethanes. The relative molecular mass of the homopolymers is generally between 1000 and 100000, that of the copolymers is between 2000 and 200000, preferably 50000 to 120000, based on the free acid, of particular suitability are also water-soluble polyacrylates which are crosslinked, for example, with about 1% of a polyallyl ether of sucrose and which have a relative molecular mass above one million. Examples thereof are the polymers available under the name Carbopol 940 and 941. The crosslinked polyacrylates are used in amounts not above 1% by weight, preferably in amounts of from 0.2 to 0.7% by weight.

The agents according to the invention can contain, as foam inhibitors, fatty acid alkyl ester alkoxylates, organopolysiloxanes and mixtures thereof with microfine, optionally silanized silica, and also paraffins, waxes, microcrystalline waxes and mixtures thereof with silanized silica. Mixtures of different foam inhibitors can also be used advantageously, e.g. those of silicone oil, paraffin oil or waxes. Preferably, foam inhibitors are bonded to a granular, water-soluble or -dispersible carrier substance.

The liquid washing agents can contain optical brighteners, for example derivatives of diaminostilbenedisulfonic acid or alkali metal salts thereof which can easily be incorporated into the dispersion. The maximum content of brighteners in the agents according to the invention is 0.5% by weight, amounts of from 0.02 to 0.25% by weight preferably being used.

The desired viscosity of the agents can be established by adding water and/or organic solvents or by adding a combination of organic solvents and further thickeners. In principle, suitable organic solvents are all mono- or polyhydric alcohols. Preference is given to using alcohols having 1 to 4 carbon atoms, such as methanol, ethanol, propanol, isopropanol, straight-chain and branched butanol, glycerol and mixtures of said alcohols. Further preferred alcohols are polyethylene glycols with a relative molecular mass below 2000. In particular, a use of polyethylene glycol with a relative molecular mass between 200 and 600 and in amounts up to 45% by weight and of polyethylene glycol with a relative molecular mass between 400 and 600 in amounts of from 5 to 25% by weight is preferred. An advantageous mixture of solvents consists of monomeric alcohol, for example ethanol and polyethylene glycol in the ratio 0.5:1 to 1.2:1, where the liquid washing agents according to the invention can contain 8 to 12% by weight of such a mixture.

Further suitable solvents are, for example, triacetin (glycerol triacetate) and 1-methoxy-2-propanol.

The thickeners used are preferably hydrogenated castor oil, salts of long-chain fatty acids, preferably in amounts of from 0 to 5% by weight and in particular in amounts of from 0.5 to 2% by weight, for example sodium, potassium, aluminum, magnesium and titanium stearates or the sodium and/or potassium salts of behenic acid, and also polysaccharides, in particular xanthan gum, guar guar, agar agar, alginates and tyloses, carboxymethylcellulose and hydroxyethylcellulose, also relatively high molecular weight polyethylene glycol mono- and diesters of fatty acids, polyacrylates, polyvinyl alcohol and polyvinylpyrrolidone.

Suitable enzymes are those from the class of proteases, lipases, amylases and mixtures thereof. Their fraction may be 0.2 to 1% by weight. The enzymes can be adsorbed onto carrier substances and/or embedded in coating substances.

Suitable acidic additives are organic or inorganic acids, preferably organic acids, particularly preferably alpha-hydroxyacids and acids selected from glycolic acid, lactic acid, citric acid, tartaric acid, mandelic acid, salicylic acid, ascorbic acid, pyruvic acid, oligooxamono- and dicarboxylic acids, fumaric acid, retinoic acid, aliphatic and organic sulfonic acids, benzoic acid, kojic acid, fruit acid, malic acid, gluconic acid, galacturonic acid, acid plant and/or fruit extracts and derivatives thereof.

In order to bind traces of heavy metals, the salts of polyphosphoric acids can be used, such as 1-hydroxyethane-1,1-diphosphonic acid (HEDP) and diethylenetriaminepentamethylenephosphonic acid (DTPMP), preferably amounts by weight of from 0.1 to 1.0% by weight.

Suitable preservatives are, for example, phenoxyethanol, formaldehyde solution, parabens, pentanediol or sorbic acid.

Suitable pearlizing agents are, for example, glycol distearic acid esters, such as ethylene glycol distearate, but also fatty acid monoglycol esters.

The salts or extenders used are, for example, sodium sulfate, sodium carbonate or sodium silicate (waterglass).

Typical individual examples of further additives are sodium borate, starch, sucrose, polydextrose, stilbene compounds, methylcellulose, toluene sulfonate, cumene sulfonate, soaps and silicones.

The agents according to the invention are usually adjusted to a pH in the range 2 to 12, preferably pH 2.1 to 7.8, particularly preferably 2.2 to 6.5.

The examples and applications below are intended to illustrate the invention in more detail without, however, limiting it thereto (all of the percentage data are in % by weight).

### EXAMPLE 1

### Solid Standard Washing Agent with C₁₂₋₁₅-oxo Alcohol Ethoxylate(8 EO)

| | |
|---|---|
| Marlon ARL | 13.0 |
| Genapol Oa 080 | 5.9 |
| Sodium stearate | 2.0 |
| Wacker ASP 15 | 5.0 |
| Zeolite A | 36.8 |
| Soda | 13.4 |
| Sokalan CP5 | 5.9 |
| 3 NaG | 3.8 |
| Tylose CR 1500 P2 | 1.5 |
| Dequest 2066 | 3.5 |
| Tinopal DMS high-conc. | 0.3 |
| Sodium sulfate | 8.5 |
| Termamyl 60T | 0.2 |
| Savinase 6.0T | 0.2 |

### EXAMPLE 2

### Solid Standard Washing Agent with C₁₂₋₁₅-oxo Alcohol Ethoxy(8 EO) Propoxylate(4 PO)

| | |
|---|---|
| Marlon ARL | 13.0 |
| Genapol EP 2584 | 5.9 |
| Sodium stearate | 2.0 |
| Wacker ASP 15 | 0.0 |
| Zeolite A | 36.8 |
| Soda | 13.4 |
| Sokalan CP5 | 5.9 |
| 3 NaG | 3.8 |
| Tylose CR 1500 P2 | 1.5 |
| Dequest 2066 | 3.5 |
| Tinopal DMS high-conc. | 0.3 |
| Sodium sulfate | 8.5 |
| Termamyl 60T | 0.2 |
| Savinase 6.0T | 0.2 |

### EXAMPLE 3

### Solid Standard Washing Agent with C₁₂₋₁₅-oxo Alcohol Ethoxy(2 EO) Propoxylate(5 PO)

| | |
|---|---|
| Marlon ARL | 13.0 |
| Genapol EP 2552 | 5.9 |
| Sodium stearate | 2.0 |
| Wacker ASP 15 | 0.0 |
| Zeolite A | 36.8 |
| Soda | 13.4 |
| Sokalan CP5 | 5.9 |
| 3 NaG | 3.8 |
| Tylose CR 1500 P2 | 1.5 |
| Dequest 2066 | 3.5 |
| Tinopal DMS high-conc. | 0.3 |
| Sodium sulfate | 8.5 |
| Termamyl 60T | 0.2 |
| Savinase 6.0T | 0.2 |

### Preparation:

I Zeolite, sodium stearate, Wacker ASP 15, soda, 3 NaG and tylose were initially introduced into a Lödige mixer at room temperature and mixed for 15 minutes.
II Genapol OA 080 or Genapol EP 2552 or Genapol 2584 was melted at 70° C. and sprayed onto I. Wacker ASP 15 was sprayed onto I at room temperature.
III Dequest 2066 was sprayed onto II at RT.
IV Mixing of Tinopal DMS-X with sodium sulfate in a multimixer.
V Addition to IV with stirring.
VI Addition of Marion ARL, Termamyl and Savinase.

### Washing Conditions:

| | |
|---|---|
| Washing machine: | Miele Novotronic W927 WPS |
| Ballast fabric: | 3.0 kg; (2 × bed sheets 1.50 × 2.60 m, 4 × |
| | pillows 0.80 × 0.80 m, standard fabric according |
| | to ISO 2267) |
| Test method: | single-liquor method |
| Water hardness: | 15° German hardness |
| Temperature: | 40° C. |
| Wash cycle: | 3× |
| Dosage: | 130 g tel quel |
| Soilings: | stain monitor Empa 102 |

### Washing Results

Difference in reflectance compared with unwashed

| | Example 1 | Example 2 | Example 3 |
|---|---|---|---|
| Earth | 25.7 | 36.1 | 43.3 |
| Grease, dyed red | 30.0 | 32.9 | 38.0 |
| Baby food | 27.2 | 27.0 | 29.8 |
| Clay earth | 23.4 | 23.9 | 26.4 |
| Butter | 40.7 | 41.2 | 44.1 |
| Engine oil | 28.5 | 31.5 | 36.0 |
| Lipstick | 20.8 | 24.2 | 31.6 |
| Make-up | 11.5 | 11.1 | 14.7 |
| Pigment/lanolin | 13.3 | 15.2 | 21.6 |
| Pigment/sebum | 19.6 | 20.8 | 24.2 |

The results show a clearly improved washing power of C_{12/15}-oxo alcohol ethoxylate(2 EO) propoxylate(5 PO) compared to C_{12/15}-oxo alcohol ethoxylate(8 EO) and compared to C_{12/15}-oxo alcoholethoxylate(8 EO) propoxylate(4 PO). When using EO/PO fatty alcohol alkoxylates, the antifoam Wacker ASP 15 can be dispensed with.

### EXAMPLE 4

### Liquid Washing Agent

| | | |
|---|---|---|
| A | Potassium hydroxide | 1.5 |
| | Edenor KPK 1218 | 5.5 |
| B | Water | ad 100 |
| C | Hostapur SAS 60 | 8.7 |
| D | Genapol EP 2552 | 10.1 |
| | 1,2-Propanediol | 1.0 |

### Preparation:

I Addition of KOH to Edenor KPK 1218
II Addition of B to I with stirring at about 30° C.
III Heating of C to about 60° C.
IV Successive addition of the components of D to III

### EXAMPLE 5

### Dishwashing Agent

| | | |
|---|---|---|
| A | Water, dist. | 18.0% |
| | Ethanol | 2.8% |
| B | ®Hostapur SAS 60 | 20.0% |
| | ®Genapol LRO liquid | 42.9% |
| | ®Genapol EP 2552 | 3.0% |
| | ®Genagen CAB 44 | 13.3% |
| | Perfume, dye, preservative | q.s. |

### Preparation:

Successive dissolution of the components of B into A with stirring

### EXAMPLE 6

### All-Purpose Cleaner

| | | |
|---|---|---|
| A | Water, dist. | 87.0% |
| B | ®Hostapur SAS 60 | 3.3% |
| | ®Genapol EP 2552 | 4.0% |
| C | Sodium cumenesulfonate (c = 40%) | 5.0% |
| | Perfume, dye, preservative | 0.7%. |

Preparation:
I Dissolution of the components of B into A with stirring
II Addition of C to I

### INCI Name of the Commercial Products Used

| | |
|---|---|
| Marlon ARL | linear sodium alkylbenzenesulfonate |
| Genapol OA 080 | C_{12/15}-oxo alcohol 8 EO adduct |
| Genapol EP 2552 | C_{12/15}-oxo alcohol 5 PO-2 EO adduct |
| Genapol EP 2584 | C_{12/15}-oxo alcohol 4 PO-8 EO adduct |
| Wacker ASP 15 | polydimethylsiloxane + filler on inorg./org. carrier |
| Sokalan CP5 | copolymer based on: maleic acid, acrylic acid, |
| | sodium salt |
| 3 NaG | sodium silicate |
| Tylose CR 1500 P2 | sodium carboxymethylcellulose |
| Dequest 2066 | diethylenetriaminepenta(methylenephosphonic acid), |
| | sodium salt |
| Tinopal DMS high | bis(triazinylamino)stilbenedisulfonic acid derivative |
| conc. | |
| Termamyl 60T | alpha-amylase (bacterial) |
| Savinase 6.0T | protease (subtilisin) |
| Edenor KPK 1218 | fatty acid |
| Hostapur SAS 60 | secondary alkanesulfonate |

1. A washing and cleaning agent comprising a fatty alcohol ethoxylate propoxylate of the formula (1)
R¹-A-OR² (1)
in which R¹ is C₁₂-C₁₅-alkyl,
A is a group of the formula
—(OC₃H₆)_{y}—(OC₂H₄)ₓ—
R² is hydrogen, x=2 and y=5.

2. The washing and cleaning agent as claimed in claim 1, which is in solid form selected from the group consisting of a powder, a granule, and a tablet, or in liquid form selected from the group consisting of an aqueous, an aqueous/organic, an aqueous alcoholic and an organic formulation.

3. The washing and cleaning agent as claimed in claim 1, wherein said agent is in the form selected from the group consisting of a solid washing agent, a liquid washing agent, a machine dishwashing agent, a hand dishwashing agent, a liquid all-purpose cleaner, a bath cleaner, a glass cleaner and a floor cleaner.

4. The washing and cleaning agent as claimed in claim 1, wherein said agent is in the form of a solid washing agent and comprises the fatty alcohol ethoxylate propoxylate of formula (1) in amounts by weight of from 0.5 to 10%.

5. The washing and cleaning agent as claimed in claim 1, wherein said agent is in the form of a liquid washing agent and comprises the fatty alcohol ethoxylate propoxylate of formula (1) in amounts by weight of from 0.5 to 10%.

6. The washing and cleaning agent as claimed in claim 1, wherein said agent is in the form of an all-purpose cleaner and comprises the fatty alcohol ethoxylate propoxylate of formula (1) in amounts by weight of from 0.5 to 10%.

7. The washing and cleaning agent as claimed in claim 1, wherein said agent further comprises a further non-ionic surfactant selected from the group consisting of an alcohol ethoxylate, an alcohol-EO/PO adduct, and mixtures thereof wherein the alcohol-EO/PO adduct has 1 to 50 mol of EO and 1 to 20 mol of PO.
